(19) United States
(12) Patent Application Publication
(10) Pub. No.: US 2005/0004403 A1
(43) Pub. Date: Jan. 6, 2005

(54) Method for producing polyether alcohols

(76) Inventors: Bernd Guttes, Sallgast (DE); Kathrin Harre, Dresden (DE); Gottfried Knorr, Ostercappeln (DE); Marita Schuster, Senftenberg (DE); Monika Wetterling, Senftenberg (DE)

Correspondence Address: BASF Corporation;Patent Department, 1609 Biddle Avenue, Wyandotte, MI, 48192, US

(21) Appl. No.: 10/495,199

(22) PCT Filed: Nov. 8, 2002
(86) PCT No.: PCT/EP02/12493

(30) Foreign Application Priority Data
Nov. 15, 2001 (DE) 101 56 014.1

(51) Int. Cl. C08G 18/48; C07C 41/03

(52) U.S. Cl. 568/679; 528/76

(57) ABSTRACT

The present invention relates to a process for the production of polyether alcohols by catalytic addition of alkylene oxides to H-functional initiators using amines as catalysts, wherein the addition of the amines to the reaction mixture is effected prior to or at the commencement of the chemical addition of the alkylene oxides and also at least once during the reaction, said additional addition of the catalyst being carried out at the point of the reaction at which there is an increased occurrence of side reactions and/or when the alkylene oxides undergo a change in the polyether chain.

The invention relates to a process for the production of polyether alcohols by reaction of H-functional initiators with alkylene oxides.

The synthesis of polyether alcohols by the reaction of H-functional initiators, particularly alcohols and primary and/or secondary amines, with alkylene oxides is well-known. The reaction of the alkylene oxides with the H-functional initiators is usually carried out in the presence of catalysts, for example, basic or acid substances or multi-metal cyanide catalysts. The catalyst used is in most cases potassium hydroxide, which is separated from the polyether alcohol, following synthesis, by purifying operations such as neutralization, distillation, or filtration. Only these pure polyether polyols are used for reaction with di- and/or poly-isocyanates to produce polyurethanes. It is also known to use amine substances such as triethylamine or, as described in WO 9,825,878, alkanolamines as catalysts. The isolation of these substances from the polyether alcohol on an industrial scale is usually difficult. However, traces of these amines used as catalysts frequently interfere with the subsequent conversion of the polyether alcohols to polyurethanes.

U.S. Pat. No. 3,346,557 describes a process for the production of polyether alcohols in which the initiator used is a mixture of solid alcohols and liquid amines. The amine serves in this case both as solvent for the solid alcohols and as catalyst for the chemical addition of the alkylene oxides. In one embodiment of this process, a prepolymer of the solid alcohol and the alkylene oxide in the presence of the amines is produced in a first step, which prepolymer is caused to react, in a second step, with alkylene oxides and with more solid alcohol and more amine. The amine is added to the reaction mixture at the start of each stage.

U.S. Pat. No. 4,228,310 describes a process for the production of polyether alcohols suitable for the synthesis of polyisocyanurate foams. In order to avoid the use of alkaline catalysts, whose degradation products are very troublesome when use is made of isocyanurate catalysts, the catalysts used are carbamate salts, aminophenols, hexahydrotriazines, and tetrahydrooxadiazines. The catalysts are added as a single batch at the commencement of the chemical addition of the alkylene oxides. Since the compounds used as catalysts also act as catalysts for the formation of isocyanurate, they can remain in the product following the synthesis of the polyether alcohols.

Since, in the process according to U.S. Pat. No. 3,346,557 and U.S. Pat. No. 4,228,310, the addition of the amines is effected irrespective of the rate of the overall reaction and, in particular, irrespective of the side reactions taking place during chemical addition of the alkylene oxides, the optimal amount of catalyst for the respective process step is not always available and momentary overcatalyzation or possibly undercatalyzation increases the formation of by-products or causes chain termination.

It is an object of the present invention to provide a process for the production of polyether alcohols using amine catalysts, which process gives high space-time yields and avoids side reactions as far as possible and in which the catalysts remain in the polyether alcohol following the reaction and can act as catalysts when use is made of said polyether alcohols for the production of polyurethanes.

In the present invention, this object is achieved by the use of amines as catalysts in the production of polyether alcohols by reaction of alkylene oxides with H-functional initiators, said amines being added prior to or at the commencement of the chemical addition of the alkylene oxides and also at least once during the reaction, which additional addition of the catalyst is effected at the point of the reaction at which there is a strong occurrence of side reactions, and/or when the alkylene oxides undergo a change in the polyether chain.

The present invention relates to a process for the production of polyether alcohols by catalytic addition of alkylene oxides to H-functional initiators using amines as catalysts, wherein the addition of the amines to the reaction mixture is effected prior to or at the commencement of the chemical addition of the alkylene oxides and also at least once during the reaction, said additional addition of the catalyst being carried out at the point of the reaction at which there is a strong occurrence of side reactions and/or when the alkylene oxides undergo a change in the polyether chain.

The invention also relates to the polyether alcohols produced by the process of the invention.

The present invention also relates to a method of using the polyether alcohols of the invention for the production of polyurethanes.

The present invention also relates to a process for the production of polyurethanes by reaction of
a) polyisocyanates with
b) compounds having at least two isocyanate-reactive hydrogen atoms,
   wherein the compounds b) having at least two isocyanate-reactive hydrogen atoms comprise at least one polyether alcohol of the invention.

Since the formation of aldehyde during the chemical addition of the alkylene oxides is a readily measurable indication of the occurrence of side reactions, the addition of the catalyst can be effected in direct relation thereto. In order to suppress side reactions effectively, the addition of the catalyst must take place before the rate of formation of aldehyde exceeds the value of 100 ppm of aldehyde/100 g of rise in molecular weight.

The concentration of aldehydes in the reaction mixture can be readily determined as a routine measure in the commercial production of polyether alcohols.

Moreover, amine catalyst is also added to the reaction mixture when there is a change in the alkylene oxides, for example, from propylene oxide to ethylene oxide and vice versa, when there is a change in the metering of an alkylene oxide to a statistical mixture of two or more alkylene oxides, and/or when there is a change in the mutual proportions of the alkylene oxides in a statistical mixture.

Amines which can be used in the process of the invention as catalysts are aliphatic and/or aromatic amines containing primary, secondary, or tertiary amino groups. Also particularly suitable are amines having a ring-shaped structure in which the nitrogen atom is enclosed in the ring.

Preferably, the ring-shaped amines used are piperazine derivatives such as 1,4-dimethylpiperazine, N-hydroxyethylpiperazine, 1,3,5-tris(dimethylaminopropyl)hexahydrostriazine, and/or N,N-dimethylcyclohexylamine, dimethylbenzylamine and/or 2,2′-bis(2-ethyl-2-azobicycloether) and/or 1,8-diazabicyclo-(5,4,0)-undecene-7 and/or morpholine derivatives such as 4-methyl- and/or 4-ethyl-morpholines and/or 2,2-dimorpholinoethyl ether and/or imidazole derivatives such as 1-methyl- and/or 1,2-dimethyl-imidazoles and/or N-(3-aminopropyl)imidazole, diazobicyclooctane (marketed under the trade name Dabco®, sold by Air Products), triethylamine, dimethylaminopropylamine, diethylaminoethylamine or arbitrary mixtures of at least two of said amines. Preferably, those amines are used which are usually employed as catalysts for the synthesis of polyurethanes, particularly imidazoles and/or diazobicyclooctane and its derivatives. The catalysts used can also include the conversion products of said amines with alkylene oxides, particularly ethylene oxide and/or propylene oxide, and more preferably propylene oxide. These conversion products preferably have a molar mass ranging from 160 to 500 g/mol.

The amines used in the process of the invention and containing primary and secondary amino groups or hydroxyl groups not only act as catalysts during the production of polyether alcohols. The free hydrogen atoms thereof can likewise gain alkylene oxides. Thus they also act as initiators in the process of the invention. The chemical addition of alkylene oxides to the free hydrogen atoms in the amino groups of the amines used causes said amino groups to be converted to tertiary amino groups.

In a particularly preferred embodiment of the process of the invention, use is made of amines exhibiting at least one tertiary amino group and at least one reactive hydrogen atom in the molecule. The reactive hydrogen atoms may, preferably, come from primary and/or secondary amino groups and/or hydroxyl groups. Since alkylene oxides also add to these reactive hydrogen atoms and the resulting polyether chains carry hydroxyl groups at the chain end, these compounds act as incorporatable catalysts during formation of the polyurethane. The advantage of incorporatable catalysts consists in that they are incorporated in the polyurethane matrix and thus cannot diffuse out of the foam. Diffusion of the polyurethane catalysts from the foams is undesirable, since they usually have a strong odor and high fogging and VOC values. By VOC value is meant the concentration of volatile or ganic components.

Examples of compounds having tertiary amino groups and reactive hydrogen atoms are N-(2-hydroxyethylmorpholine), N-3-(aminopropyl)imidazole, dimethylaminopropylamine, and diethylaminoethylamine.

The amines used in the process of the invention are employed during the production of the polyether alcohols preferably in a concentration of from 0.01 to 50 g and particularly from 0.2 to 2 g, per 100 g of initiator.

In the process of the invention, polyether alcohols, in particular, may be used for the synthesis of flexible polyurethane foams and rigid polyurethane foams.

During the production of the polyether alcohols used for the synthesis of flexible polyurethane foams, the initiators used are usually alcohols having 2 or 3 hydroxyl groups. Preferred initiators are glycerol, trimethylol propane, ethylene glycol, diethylene glycol, propylene glycol, and dipropylene glycol, and also arbitrary mixtures of at least two of said alcohols. The alkylene oxides used are mostly ethylene oxide and propylene oxide alone or together. When use is made of mixtures of ethylene oxide and propylene oxide, the alkylene oxides can be added individually in succession in so-called blocks or intermixed as a so-called statistical mixture. For certain fields of application, for example, for the synthesis of cold-mold foams, an ethylene oxide block can be added to the end of the polyether chain. The polyether alcohols used for the synthesis of flexible polyurethane foams, usually have a molecular weight Mₙ in the range of from 1,000 to 10,000 g/mol and in particular from 1,000 to 7,000 g/mol.

In the case of polyether alcohols used in the production of rigid polyurethane foams, the initiators used are mostly those having at least 4 active hydrogen atoms, preferably at least tetrafunctional alcohols and/or amines having at least 4 reactive hydrogen atoms. Both aliphatic and aromatic amines can be used. Preference is given to aromatic amines.

Examples of at least tetrafunctional alcohols are sugar alcohols, such as glucose, sorbitol, sucrose, and mannitol. Since these compounds are usually solid, the reaction thereof with the alkylene oxides is usually carried out in admixture with liquid compounds, such as water, glycerol, and/or ethylene glycol. In the process of the invention it is theoretically also possibly to use, as initiator, mixtures of said solid compounds with the amines used in the process of the invention.

The aromatic amines mostly used are toluylenediamine, diphenylmethanediamine, and mixtures of diphenylmethanediamine and polyphenylene-polymethylene-polyamines. The aliphatic amines mostly used are 1,2-diaminoethane, diethylenetriamine, dimethylpropylamine, or their higher homologs.

The reaction of the initiator with the alkylene oxides is carried out under conventional pressures ranging from 0.1 to 1.0 MPa and at conventional temperatures ranging from 80° and 140° C. Metering of the alkylene oxides is mostly followed by a post-reaction phase to complete the reaction of the alkylene oxides. In an advantageous embodiment of the process of the invention, amine catalyst is again added to the reaction mixture at the commencement of the post-reaction phase, preferably immediately on conclusion of metering of the alkylene oxides.

Following chemical addition of the alkylene oxides, the polyether alcohols are mostly subjected to a short treatment, by distillation, to remove the highly volatile impurities. If necessary, the polyether alcohol can then be filtered in order to remove any solid impurities present. It can then be processed by reaction with polyisocyanates to form polyurethanes.

Production of the polyurethanes, particularly the polyurethane foams using polyether alcohols produced by the process of the invention is carried out by known methods by reaction with polyisocyanates, mostly in the presence of catalysts, expanding agents, and, optionally, chain-extenders, crosslinking agents, and auxiliaries and/or additives.

The starting materials and auxiliaries and/or additives used will now be detailed below.

The polyisocyanates used can be aliphatic or, preferably, aromatic di- and/or poly-isocyanates. For the production of flexible polyurethane foams it is usual to use diisocyanates, particularly toluylene-diisocyanate (TDI) and diphenylmethane diisocyanate (MDI) individually or intermixed or in admixture with polyfunctional polyisocyanates.

For the production of rigid polyurethane foams, use is preferably made of polyisocyanates having a functionality of two or more. Preference is given to mixtures of diphenylmethane diisocyanate and polyphenylene-polymethylene-poyisocyanates, frequently also referred to as crude MDI.

For certain end uses it is advantageous to modify the polyisocyanates by the insertion of groups, for example, urethane, allophanate, or isocyanurate groups.

The compounds used which have at least two isocyanate-reactive hydrogen atoms are polyether alcohols of the invention alone or in admixture with other compounds having at least two isocyanatereactive hydrogen atoms.

In a preferred embodiment, the other compounds having at least two isocyanate-reactive hydrogen atoms are polyether polyols. These are produced by known methods from one or more alkylene oxides containing from 2 to 4 carbons in the alkylene radical, for example, by anionic polymerization using alkali hydroxides or alkali alkoxides as catalysts and with the addition of at least one initiator containing 2 or 3 bonded reactive hydrogen atoms. Suitable alkylene oxides are, for example, tetrahydrofuran, 1,3-propylene oxide, [1, 2 or 2,3]-butylene oxide, and preferably ethylene oxide and 1,2-propylene oxide. The alkylene oxides can be used individually, successively, or intermixed. Preference is given to mixtures of 1,2-propylene oxide and ethylene oxide, in which case the ethylene oxide is used in concentrations of from 10 to 50% as an ethylene oxide end block so that the resulting polyalcohols contain primary OH end groups to an extent of more than 70%.

Suitable initiators are water or di- and tri-hydric alcohols, such as ethylene glycol, [1,2 and 1,3]-propanediols, diethylene glycol, dipropylene glycol, 1,4-butanediol, glycerol, trimethylol propane, etc. The polyether polyols, preferably polyoxypropylenepolyoxyethylene polyols, possess a functionality of 2 or 3 and molecular weights of from 1,000 to 8,000, preferably from 2,000 to 7,000.

Other suitable polyetherols are polymer-modified polyether polyols, preferably graft polyoxyalkylene glycols, particularly those based on styrene and/or acrylonitrile, produced by in situ polymerization of acrylonitrile or styrene, or preferably mixtures of styrene and acrylonitrile.

Also suitable are polyester polyols. These can be produced, for example, from organic dicarboxylic acids containing from 2 to 12 carbons, preferably aliphatic dicarboxylic acids containing from 4 to 6 carbons, polyhydroxylic alcohols, preferably diols, containing from 2 to 12 carbons, preferably from 2 to 6 carbon atoms. Suitable dicarboxylic acids are, for example, succinic acid, glutaric acid, adipic acid, suberic acid, azelaic acid, sebacic acid, decanedioic acid, maleic acid, fumaric acid, phthalic acid, isophthalic acid, and terephthalic acid. The dicarboxylic acids can be used for this purpose either individually or intermixed. Instead of the free dicarboxylic acids, use may also be made of the corresponding dicarboxylic derivatives, such as dicarboxylates of alcohols containing from 1 to 4 carbons or dicarboxylic anhydrides. Preference is given to mixtures of dicarboxylic acids comprising succinic, glutaric and adipic acids, and aromatic dicarboxylic acids. Examples of dihydric and trihydric alcohols, particularly diols are: ethanediol, diethylene glycol, [1, 2 or 1,3]-propanediol, dipropylene glycol, 1,4-butanediol, 1,5-pentanediol, 1,6-hexanediol, 1,10-decanediol, glycerol, and trimethylol propane, further also di-alcohols containing aromatic or aliphatic ring systems, such as 1,4-bisdihydroxymethylbenzene or 1,4-bisdihydroxyethylbenzene. Preference is given to ethanediol, diethylene glycol, 1,4-butanediol, 1,5-pentanediol, and 1,6-hexanediol. Use may also be made of polyester polyols of lactones, eg, ε-caprolactone or hydroxycarboxylic acids, eg, ω-hydroxycaproic acid. It is also possible to use mixed systems containing both polyesterols and polyetherols.

In a special embodiment of the process of the invention for the production of polyurethanes, the compounds having at least two isocyanate-reactive hydrogen atoms comprise mixtures of the aforementioned conventional polyalcohols and polyether alcohols having tertiary amino groups, produced by reaction of amines selected from the aforementioned group comprising piperazine derivatives such as 1,4-dimethylpiperazine, N-hydroxyethylpiperazine, 1,3,5-tris(dimethylaminopropyl)hexahydrostriazine, and/or N,N-dimethylcyclohexylamine, dimethylbenzylamine and/or 2,2′-bis(2-ethyl-2-azobicycloether) and/or 1,8-diazabicyclo-(5,4,0)-undecene-7 and/or morpholine derivatives such as 4-methyl- and/or 4-ethyl-morpholines and/or 2,2-dimorpholinoethyl ether and/or imidazole derivatives such as 1-methyl- and/or 1,2-dimethyl-imidazoles and/or N-(3-aminopropyl)imidazole, diazobicyclooctane, triethylamine, dimethylaminopropylamine, diethylaminoethylamine, or arbitrary mixtures of at least two of said amines with alkylene oxides. Without other initiators being present, the amines are caused to react with alkylene oxides, and the resulting polyether alcohols are mixed with other polyether alcohols prior to reaction with the polyisocyanates.

In order to produce the polyurethanes, use is frequently made of chain extenders and/or crosslinking agents. The chain extenders and crosslinking agents used are mostly alcohols having a functionality of two or more or amines having molecular weights ranging from 60 to 400 g/mol.

The expanding agent used is preferably water, which reacts with the isocyanate groups with elimination of carbon dioxide, and/or a compound which is inert to the starting compounds of the polyurethane reaction and which vaporizes due to the heat of reaction generated during formation of the polyurethane, a so-called physical expanding agent. Examples of physical expanding agents are aliphatic hydrocarbons containing from 3 to 8 carbons, particularly pentanes, halogenated hydrocarbons, or acetals. Another possibility is the use of gases which dissolve in the starting compounds under pressure, for example carbon dioxide, nitrogen, or noble gases, as expanding agent.

As mentioned above, the amines used as catalysts for the production of the polyether alcohols also act as catalysts for the production of polyurethane. For certain fields of application it is possible to use additional catalysts for the production of polyurethane, particularly compounds having tertiary amino groups and/or organic metal complexes, particularly tin compounds. Said catalysts may be the aforementioned conversion products of amines used as catalysts for the production of polyether alcohols with alkylene oxides, particularly ethylene oxide and/or propylene oxide and more preferably propylene oxide, having a molar mass ranging from 160 to 400 g/mol.

Auxiliaries and/or additives used are, for example, stabilizing agents, flameproofing agents, and/or pigments.

Production of the polyurethanes can be carried out by known methods, for example, by the one shot or prepolymer process; the foamed plastics can be produced by the block-foaming method or the mold-foaming method.

Further statements on the feedstocks used and on the production of the foamed plastics are to be found, for example, in Kunststoffhandbuch, Vol. 7, “Polyurethane”, Carl-Hanser-Verlag Munich, 1st Edition 1966, 2nd Edition 1983 and 3rd Edition, 1993.

The process of the invention has several advantages. As it is possible to use the same amine catalysts for the successive poly-addition reactions for the production of the polyether alcohol and for the production of the polyurethane, it is possible to dispense with elaborate purifying operations following synthesis of the polyether alcohols.

The deliberate addition of the catalysts at points of monomer change or at points of the reaction preceding any increased formation of by-products raises the space-time yield for the production of the polyether alcohols and suppresses the formation of by-products. The polyurethanes produced using polyether alcohols produced by the process of the invention show a lower tendency to fogging and are substantially inodorous. This is due to the greatly reduced amount of by-products formed and also to the capture of the catalyst in the polyurethane skeleton.

The invention is illustrated below with reference to the following examples.

### EXAMPLE 1

In an autoclave having a capacity of 1 L there were successively placed 71 g of diethylene glycol, 162 g of sucrose, and 2 g of dimethylcyclohexylamine, after which the autoclave was purged with nitrogen and heated to 110° C. Once this temperature had been reached, 100 g of ethylene oxide were metered into the stirred reaction mixture and caused to react therein. A further 5 g of dimethylcyclohexylamine were then added to the reaction mixture. After the addition of catalyst, 300 g of propylene oxide were metered in at 120° C. and caused to react. The polyether alcohol was distilled, in order to remove highly volatile impurities, over a period of two hours at 115° C. to a water content of 0.02 percent and had the following characteristics:
hydroxyl value: 441 mg KOH/g
viscosity at 25° C.: 5870 mPa•s
pH: 10.1.

### EXAMPLE 2

Production of a Rigid Polyurethane Foam

54 parts by weight of polyether alcohol of Example 1, 4.2 parts by weight of glycerol, 21.1 parts by weight of a polyether alcohol based on monoethylene glycol and propylene oxide and having a hydroxyl value of 105 mg KOH/g, one part by weight of silicone stabiliser Tegostab® B 8409, sold by Goldschmidt A G, 1.8 parts by weight of dimethylcyclohexylamine, 2.4 parts by weight of water, and 15.5 parts by weight of cyclopentane were combined to form a polyalcohol component and then vigorously mixed with 125 parts by weight of crude MDI having an NCO content of 31.5 wt %.

The foam thus produced by free foaming in a foaming beaker had a density of 29 g/L.

The compressive strength of a foam produced with these starting materials in a closed mold at 10% densification was 0.14 N/mm².

### EXAMPLE 3

In an autoclave there were successively introduced 100 g of prepolymer, produced by chemical addition of 90 g of propylene oxide to 10 g of glycerol, catalyzed using 2 g of diazobicyclooctane, and 7 g of diazobicyclooctane, after which the autoclave was purged with nitrogen and heated to 120° C. At this temperature, 350 g of propylene oxide were metered in and caused to react, and at the commencement of the chemical addition of the propylene oxide, the formation of aldehyde in analogous KOH-catalyzed syntheses of polyetherol exceeded a value of 100 ppm of aldehyde 100 g of rise in molecular weight. On conclusion of chemical addition of the propylene oxide, the polyether alcohol was freed from residual amounts of unconverted propylene oxide by stripping with nitrogen. 500 g of ethylene oxide were then metered in and caused to react. The resulting polyether alcohol had the following characteristics:
hydroxyl value: 33.5 mg KOH/g,
water content: 0.1 wt %
pH: 9.8.
   Production of Flexible Polyurethane Foams

### EXAMPLE 4

83.3 parts by weight of polyether alcohol of Example 3, 10 parts by weight of a graft polyalcohol based on styrene and acrylonitrile and having a hydroxyl value of 25 mg KOH/g, 0.5 part by weight of glycerol, 1 part by weight of a polyether alcohol based on glycerol, ethylene oxide, and propylene oxide and having a hydroxyl value of 42 mg KOH/g, 0.5 part by weight of amine catalyst Dabco® 2025, sold by Air Products, 0.5 part by weight of amine catalyst Lupragene® N 211, sold by BASF AG, 0.4 part by weight of silicone stabiliser Tegostab® 8680, sold by Goldschmidt A G, and 3.8 parts by weight of water were combined to form a polyalcohol component. This was mixed with an NCO group-containing prepolymer based on MDI and having an NCO content of 27.5 wt % at an index of 100 and was poured into an open mold and allowed to cure therein.

The properties of the resultant foam are listed in Table 1.

### EXAMPLE 5

### For Comparison

Example 4 was repeated except that instead of polyether alcohol of Example 3, 82.95 parts by weight of a polyether alcohol based on glycerol, propylene oxide, and ethylene oxide and having a hydroxyl value of 28 mg KOH/g and additionally 0.35 part by weight of amine catalyst dimethylpropyldiamine were used.

The properties of the resultant foam are listed in Table 1.

### EXAMPLE 6

### Invention

83.3 parts by weight of a polyether alcohol based on glycerol, propylene oxide, and ethylene oxide and having a hydroxyl value of 28 mg KOH/g, 10 parts by weight of a graft polyalcohol based on styrene and acrylonitrile and having a hydroxyl value of 25 mg KOH/g, 1 part by weight of a polyether alcohol based on glycerol, ethylene oxide, and propylene oxide and having a hydroxyl value of 42 mg KOH/g, 1 part by weight of a polyether alcohol based on dimethylpropyldiamine and propylene oxide and having a hydroxyl value of 324 mg of KOH/g, 0.5 part by weight of amine catalyst Lupragen® N 211, sold by BASF AG, 0.4 part by weight of silicone stablisator Tegostab® 8680, sold by Goldschmidt A G, and 3.8 parts by weight of water were combined to form a polyalcohol component. This was mixed with an NCO group-containing prepolymer based on MDI and having an NCO content of 27.5 wt % at an index of 100 and was poured into an open mold and allowed to cure therein.

The properties of the resultant foam are listed in Table 1.

### EXAMPLE 7

### For Comparison

Example 6 was repeated except that there were used, instead of 83.3 parts by weight, 82.95 parts by weight of polyether alcohol based on glycerol, propylene oxide, and ethylene oxide and having a hydroxyl value of 28 mg KOH/g, 0.5 part by weight of glycerol, 0.5 part by weight of amine catalyst Dabco® 2025, sold by Air Products, no polyether alcohol based on dimethylpropyldiamine and propylene oxide but instead 0.35 part by weight of dimethylpropyldiamine.

The properties of the resultant foam are listed in Table 1.

| Example | 4 | 5 (C) | 6 | 7 (C) |
|---|---|---|---|---|
| Bulk density (g/L) | 45 | 45 | 45 | 45 |
| Strain after fracture | 85 | 90 | 95 | 90 |
| (%) | | | | |
| Tensile strength (kPa) | 120 | 100 | 100 | 100 |
| Compression set, | 4 | 3.5 | 4.4 | 3.5 |
| 50%, 70° C., 22 h (%) | | | | |
| Compression set, | 18 | 14 | 15 | 144 |
| autoclave (%) | | | | |
| Compressive strength | 5.5 | 5.5 | 6 | 5.5 |
| 40% (kPa) | | | | |
| Fogging (mg) | 0.2 | 0.4 | 0.2 | 0.4 |
| Odor | weak | distinct | weak | distinct |
| VOC (ppm) | n.d. | n.d. | 98 | 358 |
| FOG (ppm) | n.d. | n.d. | 116 | 126 |

VOC (volatile organic chemicals) is a measure of the gaseous emission of components from the foam.
FOG is a measure of condensable emissions from the foam.
n.d.—not determined.

1. A process for the production of a polyether alcohol comprising catalytic addition of an alkylene oxide to an H-functional initiator using an amine as catalyst, wherein the addition of the amine to the reaction mixture is carried out prior to or at the commencement of the chemical addition of the alkylene oxide and also at least once during the reaction, said additional addition of the catalyst being effected at the point of the reaction at which there is an increased occurrence of side reactions and/or the alkylene oxide undergoes a change in the polyether chain.

2. A process as defined in claim 1, wherein the amine is selected from the group of 1,4-dimethylpiperazine, N-hydroxyethylpiperazine, 1,3,5-tris(dimethylaminopropyl)hexahydrostriazine, N,N-dimethylcyclohexylamine, dimethylbenzylamine, 1,8-diazabicyclo-(5,4,0)undecene-7,4-methylmorpholine, 4-ethylmorpholine, 2,2-dimorpholinoethyl ether, 1-methyl- and/or 1,2-dimethyl-imidazole, N-(3-aminopropyl)imidazole, triethylamine, 2,2′-bis(2-ethyl-2-azobicycloether), diazobicyclooctane, dimethylaminopropylamine, diethylaminoethylamine, and a mixture thereof.

3. A process as defined in claim 1, wherein the amine contains at least one tertiary amino group and at least one alkylene oxide-reactive hydrogen atom in the molecule.

4. A polyether alcohol produced in accordance with the process of any of claims 1 to 3.

5. A method of producing a polyurethane using a polyether alcohol produced in accordance with the process of any of claims 1 to 3, for the production of polyurethanes.

6. A process for the production of a polyurethane comprising the reaction product of
a) a polyisocyanate with
b) a compound having at least two isocyanate-reactive hydrogen atoms,
wherein the compound b) having at least two isocyanate-reactive hydrogen atoms is a polyether alcohol produced in accordance with the process of in any of claims 1 to 3.

7. A process for the production of a polyurethane comprising the reaction product of
a) a polyisocyanate with
b) a compound having at least two isocyanate-reactive hydrogen atoms,
wherein the compound b) having at least two isocyanate-reactive hydrogen atoms contains at least one polyether alcohol containing at least one tertiary amino group, produced by convertion of at least one amine, selected from the group comprising 1,4-dimethylpiperazine, N-hydroxyethylpiperazine, 1,3,5-tris(dimethylaminopropyl)hexahydrostriazine, N,N-dimethylcyclohexylamine, dimethylbenzylamine, 1,8-diazabicyclo-(5,4,0)undecene-7,4-methylmorpholine, 4-ethylmorpholine, 2,2-dimorpholinoethyl ether, 1-methyl- and/or 1,2-dimethyl-imidazole, N-(3-aminopropyl)imidazole, triethylamine, 2,2′-bis(2-ethyl-2-azobicycloether), diazobicyclooctane, dimethylaminopropylamine, diethylaminoethylamine, and a mixture thereof.
